(19) United States
(12) Patent Application Publication
(10) Pub. No.: US 2004/0242967 A1
(43) Pub. Date: Dec. 2, 2004

(54) Handgrip for a laryngoscope

(75) Inventors: Helmut M. Heine, Herssching (DE); Dieter Fottner, Paehl (DE); Gerhard Guegel, Diessen (DE)

Correspondence Address: ROTHWELL, FIGG, ERNST & MANBECK, P.C., 1425 K STREET, N.W., SUITE 800, WASHINGTON, DC, 20005, US

(73) Assignee: Heine Optotechnik GmbH & Co. KG, Herrsching (DE)

(21) Appl. No.: 10/854,681

(22) Filed: May 27, 2004

(30) Foreign Application Priority Data
Aug. 14, 2003 (DE) 203 12 563.0
May 28, 2003 (DE) 203 08 431.4

(51) Int. Cl. A61B 1/267

(52) U.S. Cl. 600/197

(57) ABSTRACT

A handgrip for a laryngoscope, including a housing made of plastic having an upper end face at which an attachment device for fixing a spatula containing a light guide, and an upper opening are provided, and a closure device closing an open, lower end of said housing, the closure device being movable from an OFF position to an ON position, said upper opening being closed by a transparent material.

### FIELD OF THE INVENTION

The invention relates to a handgrip for a laryngoscope with an elongate housing at whose upper end face there is an attachment device, for fixing a spatula containing a light guide, and also an upper opening, and whose lower end is open and can be closed by a closure device.

### DESCRIPTION OF THE BACKGROUND ART

A handgrip of this kind is known from German utility model 297 23 221. This handgrip has an elongate cylindrical housing of metal into which a battery is fitted. Formed at the head end of the housing is a through-opening into which a lamp is fitted which is biased upwards by a helical spring, the lamp protruding upwards out of the housing in an OFF position. Provided on the spatula is a light guide which, when the spatula is secured on the attachment device, presses against the lamp and presses the latter downwards counter to the biasing force of the spring, as a result of which the lamp is connected electrically conductively to the battery.

Laryngoscopes are used, inter alia, for examining the laryngeal region of a patient and for intubation. This can lead to contamination of the spatula and of the handgrip with blood, saliva or other body fluids. To prevent cross-contamination from one patient to the next, it is necessary to sterilize the known handgrip and the spatula before a subsequent use. However, this sterilization is very complex.

To avoid sterilization after each use, it is known from U.S. Pat. No. 4,848,558 to engage a transparent impermeable protective sleeve over the spatula and the handgrip, which protective sleeve is pulled off and discarded after examination of a patient. The protective sleeve has to be pulled off very carefully in order to avoid contamination of grip and spatula surface.

Finally, one-piece disposable laryngoscopes are known which, after a single use, are completely discarded together with all the current-carrying parts.

### SUMMARY OF THE INVENTION

It is an object of the invention to make available, by structurally simple means, a disposable handgrip for a laryngoscope, in which the parts to be disposed of after a single use can be produced inexpensively.

According to a first aspect of the invention, this object is achieved by a handgrip for a laryngoscope, comprising a housing made of plastic having an upper end face at which an attachment device for fixing a spatula containing a light guide, and an upper opening are provided, and a closure device closing an open, lower end of said housing, said closure device being movable from an OFF position to an ON position, said upper opening being closed by a transparent material.

According to a second aspect of the invention, this object is achieved by a handgrip for a laryngoscope, comprising an elongate housing made of plastic and having a closed lower end and an open upper end, a closure device which closes said open upper end of said housing and being movable from an OFF position to an ON position, an attachment device which is provided on a top face of said closure device for fixing a spatula containing a light guide, and an upper opening which is provided in said closure device and is closed by a transparent material.

In the handgrips according to the invention, the housing is completely closed after application of the closure device, so that entry of saliva, blood or other body fluids into the handgrip is prevented. After use, a light insert which has been introduced into the housing is removed, after which the handgrip no longer contains any current-carrying parts and is disposed of. The parts to be disposed of, i.e. the housing, the closure device, and a spatula which can be fixed on the handgrip, can be produced inexpensively.

### BRIEF DESCRIPTION OF THE DRAWINGS

An illustrative embodiment of the invention is explained in more detail below with reference to the drawings, in which:

FIG. 1 shows a side view of a handgrip and of a closure cap of a laryngoscope;

FIG. 2 shows the cross section A from FIG. 1;

FIG. 3 shows, in cross section, the handgrip from FIG. 2, a light insert having been fitted into the handgrip, and the closure cap being situated in an OFF position;

FIG. 4 shows the view from FIG. 3, the closure cap being situated in an ON position.

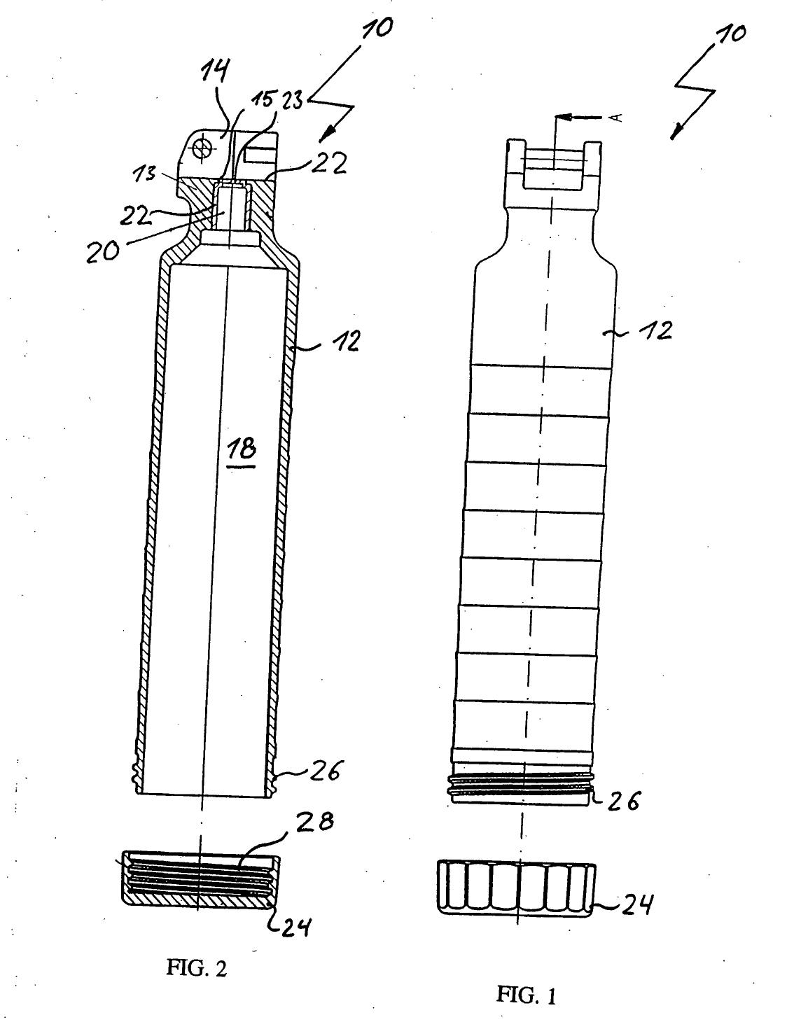

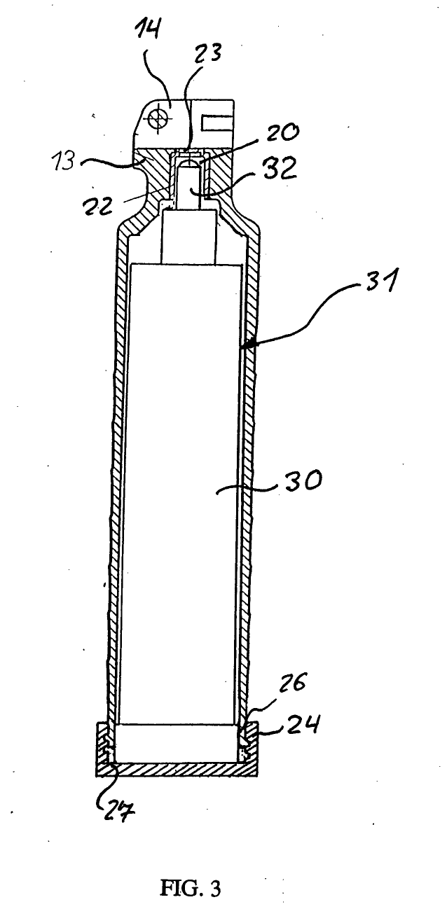

### DETAILED DESCRIPTION OF THE INVENTION

The handgrip 10 has a substantially cylindrical elongate housing 12 made of plastic which narrows at its upper head portion 13. Arranged on the end face 22 of the head portion 13 there is an attachment device 14 for fixing a spatula (not shown), which attachment device 14 is designed substantially like the attachment device known from German utility model 297 23 221.

The housing 12 has an elongate cylinder space 18 which is open at the bottom and which narrows at the head portion 13 into a cylindrical head space 20 which opens out though an opening 15 in the end face 22.

A pot-shaped insert piece 22, also made of transparent plastic and open at the bottom, is fitted into the cylinder space 20, its upper end face 23 being flush with the end face 22 and closing the opening 15.

An external thread 26 is formed at the lower end of the housing 12. A pot-shaped closure cap 24 made of plastic has an internal thread 28 and can be screwed onto the external thread 26.

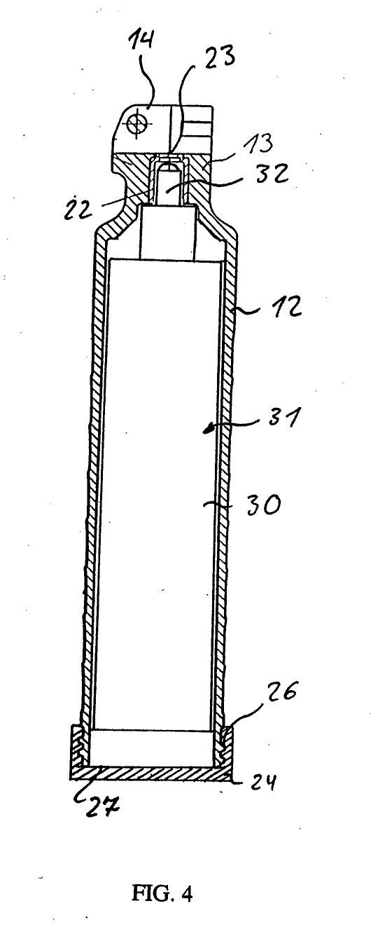

As is shown in FIGS. 3 and 4, a light insert 31 having an elongate cylindrical battery body 30 is fitted into the housing 12 at the time of use. Provided on the upper end of the battery body 30 there is a lamp 32 which extends into the insert piece 22 of transparent material. The lamp 32 is biased upwards by a biasing device (not shown) and is displaceable in the longitudinal direction of the housing 12 and of the battery body 30.

In the OFF position shown in FIG. 3, the closure cap 24 is screwed only partially onto the thread 26 of the housing 12, so that the base 27 of the closure cap 24 is situated at a distance from the lower end of the housing 12.

The length of the light insert 31 is such that the battery body 30 in the OFF position in FIG. 3 lies on the base 27 of the closure cap 24, and the lamp 32 is situated just underneath the top end face 23 of the insert piece 22, or bears on the latter, the lamp 32 in this position not being connected electrically conductively to an accumulator in the battery body 30.

In the position shown in FIG. 4, the closure cap 24 is screwed fully onto the external thread 26, so that the lower end of the housing 12 lies on the base 27. When the closure cap 24 is screwed from the OFF position shown in FIG. 3 to the ON position shown in FIG. 4, the battery body 30 is moved upwards. Since the lamp 32 comes to lie on the end face 23 of the insert piece 22 in this movement, or is already bearing on it, the battery body 30 is moved upwards relative to the lamp 32 counter to the biasing force of the biasing device, by which means the lamp 32 is connected electrically conductively to the accumulator in the battery body 30 and lights up. The light from the lamp 32 passes through the end face 23 of the insert piece 22 and, after application of a spatula, into the light guides in the latter.

After use, the closure cap 24 is unscrewed from the housing 12, and the light insert 31 is removed and placed in a charger unit. The housing 12 and the closure cap 24 are then disposed of.

In another embodiment of the handgrip 10, the housing 12 can be closed at the lower end, and the head portion 13 can be screwed onto the upper end of the housing 12. The head portion 13 then serves as a closure device which can be moved by screwing from the OFF position to the ON position.

1. A handgrip for a laryngoscope, comprising
a housing made of plastic having an upper end face at which an attachment device for fixing a spatula containing a light guide, and an upper opening are provided, and
a closure device closing an open, lower end of said housing, said closure device being movable from an OFF position to an ON position, said upper opening being closed by a transparent material.

2. The handgrip according to claim 1, wherein said housing is cylindrical, and said closure device is formed by a pot-shaped closure cap which is open at a top thereof and which is in threaded engagement with said lower end of said housing.

3. The handgrip according to claim 1, wherein said closure cap has an internal thread which is in threaded engagement with an external thread at said lower end of said housing.

4. The handgrip according to claim 1, wherein a light insert bearing with its lower end on said closure device is fitted into said housing, said light insert having a battery device and lighting means which is arranged above said battery device and below said transparent material and which is displaceable in the longitudinal direction of said housing in relation to said battery device and is biased upwards by a biasing device, said light insert being designed in such a way that, upon movement of said closure device from said OFF position to said ON position, said lighting means bears or comes to bear on said transparent material, and said battery device is moved counter to said biasing force of said biasing device in the direction of said lighting means, as a result of which said lighting means is connected electrically conductively to said battery device.

5. Handgrip for a laryngoscope, comprising
an elongate housing made of plastic and having a closed lower end and an open upper end,
a closure device which closes said open upper end of said housing and being movable from an OFF position to an ON position,
an attachment device which is provided on a top face of said closure device for fixing a spatula containing a light guide, and
an upper opening which is provided in said closure device and is closed by a transparent material.

6. Handgrip according to claim 5, wherein a light insert bearing with its lower end on said lower end of said housing is fitted into said housing, said light insert having a battery device and also lighting means which is arranged above said battery device and below said transparent material and which is displaceable in the longitudinal direction of said housing in relation to said battery device and is biased upwards by a biasing device, said light insert being designed in such a way that, upon movement of said closure device from said OFF position to said ON position, said lighting means bears or comes to bear on said transparent material, and said battery device is moved counter to said biasing force of said biasing device in the direction of said lighting means, as a result of which said lighting means is connected electrically conductively to said battery device.
